(19) United States
(12) Patent Application Publication
(10) Pub. No.: US 2024/0285490 A1
(43) Pub. Date: Aug. 29, 2024

(54) ECOBIOLOGICAL LIPO-ALCOHOLIC COMPOSITION

(71) Applicants: Jean-Noel THOREL, PARIS (FR); NAOS INSTITUTE OF LIFE SCIENCE, Aix En Provence (FR)

(72) Inventor: Jean-Noel THOREL, Paris (FR)

(21) Appl. No.: 18/000,863

(22) PCT Filed: Sep. 3, 2020
(86) PCT No.: PCT/EP2020/074563

§ 371 Date: Apr. 5, 2023

(30) Foreign Application Priority Data
Jun. 8, 2020 (FR) FR2005970

(51) Int. Cl. A61K 8/34; A61K 8/04; A61K 8/06; A61K 8/22; A61K 8/49; A61K 8/60; A61Q 17/00

(52) U.S. Cl. CPC A61K 8/34; A61K 8/042; A61K 8/062; A61K 8/22; A61K 8/342; A61K 8/498; A61K 8/602; A61Q 17/005; A61K 2800/30; A61K 2800/33

(57) ABSTRACT

The invention relates to an eco-biological, lipo-alcoholic topical disinfectant composition that is virucidal, where appropriate bactericidal and/or yeasticidal, by friction and without rinsing, capable of regenerating the skin lipid barrier and perfectly well tolerated by the skin, including during repeated daily intensive use, characterized in that it comprises: at least 65% w/w of a C₁-C₈ alcohol; at least 10% by total weight of the composition of an oily phase comprising at least one component chosen from the list of the following components: plant oils, mineral oils, silicone oils, butters, esters, plant waxes, mineral waxes, silicone waxes, fatty acids, and fatty alcohols with fatty chains of more than 10 carbon atoms in length.

### FIELD OF THE INVENTION

The invention relates to a new category of eco-biological cosmetic topical products comprising a defined dose of alcohol in order to be effective as a virucide, bactericide and yeasticide and a defined dose of a lipid substrate making it possible to preserve and regenerate the skin lipid film, thus making it possible for said skin lipid film to maintain its barrier effect properties and its hydration. This combination is exceptionally well-tolerated, including during intensive uses (repeated multiple times during the week). The invention also relates to the use of this composition to disinfect the skin and to a method for the manufacturing of said composition.

### PRIOR STATE OF THE ART

In general, the skin contains millions of different micro-organisms (bacteria, viruses, yeasts, etc . . . ) which are present at concentrations that exceed millions, even billions, of units forming colonies (UFCs) per square centimetre (cm²).

Many of these micro-organisms are harmless, but there are also various pathenogenic types or sub-species, such as Escherichia coli and Staphylococcus aureus. Several other bacteria can be found in the skin flora (or skin microbiota), such as Staphylococcus epidermidis which is generally not pathenogenic. Hands are also instruments of viral or bacterial propagation.

For these reasons, washing and/or disinfecting the skin, in particular, the hands, is recommended by health care professionals. During periods of health crises and epidemics, cleaning one's hands is more urgently recommended since this is one of the most effective actions to repeat in order to limit transmission and/or contamination risks.

Hand washing is specifically done with washing compositions, washing gels or even by friction with hydro-alcoholic solutions or gels.

Among the traditional solutions currently used to wash/disinfect the skin, in particular, the hands, are numerous cosmetic or dermo-pharmaceutical products that can be viricidal, bactericidal or yeasticidal, such as Pevaryl®, Econazole®, Mycoapaisyl®, Betadine®, Dakin® or Exomedine®. These products are formulated either by using biocides, from a list of substances that are toxic to bacteria, yeasts or viruses, that are closely regulated in Europe, or by using a necessary and sufficient amount of alcohol, specifically isopropanol or ethanol, which can penetrate the membranes of micro-organisms and destroy them.

Biocides are particularly harsh on the skin and the skin microbiome, in other words, on the community of micro-organisms that comprise the skin flora. Therefore, biocides give rise to secondary effects such as intolerance, allergies or even skin lesions, which are frequently reported by health professionals; in general, their use is limited.

Alcohol causes a rupture or complete destruction of the hydro-lipidic film, which causes a change in the skin film and in its barrier effect, as well as very uncomfortable secondary effects. These effects are described at length in the scientific literature as occurring during intensive uses of these alcohols in the form of hydro-alcoholic solutions or gels, for example, for disinfecting the hands of hospital staff in order to avoid the propagation of viral infections (for example, the article by Lan J, et al. Skin damage among healthcare workers managing coronavirus disease-2019, Journal of the American Academy of Dermatology(2020) or the Article by Cavanagh G, Wambier C, Rational hand hygiene during the COVID-19 pandemic, Journal of the American Academy of Dermatology (2020) or the article by Yan Y et al. Consensus of Chinese experts on protection of skin and mucous membrane barrier for health-care workers fighting against coronavirus disease 2019.

Dermatol Ther. 2020 or the article by Wan Y et al. Receptor Recognition by the Novel Coronavirus from Wuhan: an analysis based on decade-long structural studies of SARS Coronavirus. 2020. J Virol 94: e00127-20).

However, the skin is the first protective barrier against outside attacks (pathogens, contamination, chemical attacks . . . ). The barrier function of the skin places it in the role of protecting against outside stress factors and also prevents the evaporation of water. When it is regularly attacked by such cleaning compositions, the skin may lose its natural defensive and protective abilities over time. Repeated washing many times a day have an unquestionable effect on the skin ecosystem by eliminating the lipids that naturally preserve and protect the skin. This is even more so the case when disinfectants are used several times a day, because their high alcohol concentration changes the skin's lipid film, which has the specific effects of making it lose its barrier effect and of causing a significant increase in the transepidermal loss of water, a strong penetration of the substances applied to its surface and more and more frequent intolerances (redness, dryness, cracking, lesions . . . ).

Cleaning products that can be used daily and repeatedly, without water, but rather by friction, are effective in eliminating viruses, bacteria and yeasts, especially those present on the surface of the hands, and therefore, in fighting against disease transmission. Nonetheless, these formulations attack the skin barrier and their use causes harmful consequences to health.

Certain formulations of the prior art (with examples given below in example 7) combine microbiocidal compositions (bactericides and/or yeasticides) and virucides that disinfect the hands and hydrate the skin. Nonetheless, these compositions are poorly tolerated by human tissues and cause redness and irritation, which are uncomfortable for users, in particular, for hospital staff who must disinfect their hands often, as many as 50 times a day.

As a result, and in order to counteract the undesirable effects of hydro-alcoholic solutions or gels, users frequently apply a hydrating composition (cream, solution, lotion . . . ) after having applied the hydro-alcoholic solution or gel.

Therefore, there is an obvious need to perfect topical formulations, that are perfectly tolerated by the skin tissues, making it possible, in one simple application, to obtain, by friction, the virucidal, bactericidal and yeasticidal effects of a gel or a hydro-alcoholic solution without its problems (intolerance, dehydration, flaking, changes in the function of the skin barrier . . . ) while making it possible to reconstruct the skin barrier by improving the quality of its lipid film.

### SPECIFICATION OF THE INVENTION

The Applicant has perfected a new category of innovative products making it possible, by means of a single product called an eco-biological, lipo-alcoholic, topical formulation, to obtain the properties of several products without their associated problems. This new category of innovative products makes it possible to effectively clean the skin, for example, by effectively cleaning the hands, without the problems of the prior art. In other words, the Applicant has perfected formulations that, unexpectedly, provide three complementary properties: disinfection, hydration/maintenance of the skin barrier, and very high tolerance.

According to a first aspect, the invention relates to an eco-biological, lipo-alcoholic topical disinfectant composition that is virucidal, where appropriate bactericidal and/or yeasticidal, by friction and without rinsing, capable of regenerating the skin lipid barrier and perfectly well tolerated by the skin, including during repeated daily intensive use, characterized in that it comprises:
at least 65% w/w of a C₁-C₈ alcohol;
at least 10% by total weight of the composition of an oily phase comprising at least one component chosen from the list of the following components: plant oils, mineral oils, silicone oils, butters, esters, plant waxes, mineral waxes, silicone waxes, fatty acids, and fatty alcohols with fatty chains of more than 10 carbon atoms in length.

According to the invention, the alcohol content must be greater than or equal to 65% by weight of the composition in order to provide a virucidal effect, and if applicable, a bactericidal effect and, possibly, a yeasticidal effect.

According to the invention, the oily phase must be greater than or equal to 10% by weight of the composition in order to provide true reconstruction of the lipid film, with lower concentrations not making it truly possible to reconstruct the skin barrier effect destroyed by alcohol.

In other words, the Applicant has perfected a composition that that provides a virucidal effect, if applicable, a bactericidal effect and possibly, a yeasticidal effect, by means of friction, regenerating the hydro-lipid surface film, with no major intolerance phenomena having been observed.

According to the invention, “eco-biologic composition” means a composition that does not disturb the biology of the skin, in particular, the lipid or hydro-lipid barrier, and that is well-tolerated by the skin. Therefore, an eco-biologic composition according to the invention, makes it possible to respect the barrier property of the skin consisting of:
preventing the entry of poorly tolerated, or even toxic, components likely to generate the production of inflammatory mediators; and
limiting/preventing transepidermal water loss (TWL) despite the use of alcohol at a high concentration.

The quality of the barrier function of the skin can be measured by an in vivo measurement of transepidermal water loss (TWL). Unlike transpiration, transepidermal water loss is not visible to the naked eye. The amount of water crossing the stratum corneum (most superficial layer of the skin) by this rout is assessed at between 300 and 400 ml/d under normal conditions, but can be multiplied by 10 or 20 in the case of irritations or rupture of the barrier effect. TWL is measured by using, for example, the Tewameter TM300 (Monaderm), and the results are expressed in % reduction of the TWL (improvement in the barrier effect) in relation to average TWL values, measured after the application of a standard hydro-alcoholic solution on the market. Thus, a TWL greater than or equal to 50% indicates an improvement in the barrier effect.

According to one particular embodiment, the composition according to the invention provides a reduction of the TWL greater than or equal to 50% (Tewameter TM300 (Monaderm) of this measurement [compared] with a standard hydro-alcoholic solution on the market.

In other words, according to the invention, an eco-biologic composition provides a tolerance effect to the skin and promotes the prevention/regeneration of the skin lipid barrier.

According to the invention, “lipo-alcoholic composition” means a composition comprising an alcohol phase, an oily phase and, possibly, water. When the composition comprises water, the water concentration is less than the concentration of the oily components and of the alcohol components (water <oily phase and water<alcohol phase).

According to a specific embodiment, water represents less than 20% by total weight of the composition according to the invention, advantageously less than 15% and preferably less than 10%.

The composition according to the invention provides a virucidal effect and/or an antimicrobial effect (that is, a bactericidal and/or yeasticidal effect), advantageously a virucidal, bactericidal and yeasticidal effect, while making possible the preservation and/or restoration of the intrinsic properties of the skin, that is, it provides a barrier effect and is well-tolerated.

According to a particular embodiment, the C₁-C₈ alcohol is chosen from among the following list of components: ethanol, isopropanol, propanol, butanol, pentanol, hexanol, heptanol, octanol, each being in a linear or branched form, and their mixtures.

Advantageously, the C₁-C₈ alcohol is chosen from among the following list of components: ethanol, isopropanol, and their mixtures.

According to a particular embodiment, the eco-biological, lipo-alcoholic composition according to the invention comprises between 65% and 80% w/w of a C₁-C₈ alcohol, advantageously between 70% and 80% w/w.

According to a particular embodiment, the oily phase of the composition according to the invention comprises at least one vegetable oil from among the following list of components INCI designations): vegetable squalene, hexyl laurate, octododecanol, dibutyl adipate, coco-caprylate/caprate, dicaprylyl carbonate, decyl oleate, dicaprylyl ether, cocoglycerides, C10-18 triglyceride, caprylic/capric triglyceride, prunus oil and prunus kernal oil, ribes seed oil, pongamia seed oil, corylus seed oil, brassica seed oil, butyrospermum parkii oil, helianthus annuus seed oil, olea europaca fruit oil, coco nucifera oil, simmondsia chinensis seed oil, canola oil, cyperus esculentus root oil, limnanthes seed oil, mangifera seed butter, oryza sativa bran oil, camellia joponica seed oil, polyglycerl-3 diisostearate and their mixtures.

According to a particular embodiment, the oily phase of the composition according to the invention comprises at least one fat or oil chosen from among the list of following components (INCI designations): isohexadecane, isododecane, propylheptyl caprylate, cetearyl isononanoate, ethylhexyl hydrostearate, ethylhexyl palmitate, diisopropyl sebacate, butyloctyl salicylate, propylene glycol dicaprylate/dicaprate, tridecyl trimellitate, C12-C15 alkyl benzoate, hydrogenated polydecene, caprylyl methicone, triethylhexanoin, paraffinum liquidum/minerai oil, dipentaerythrityl hexacaprylate/hexacaprate, hydrogenated polyisobutene, dimethicone, dimethicone, caprylyl methicone and their mixtures.

According to a particular embodiment, the oil phase of the composition according to the invention comprises at least one polymer chosen from among the list of the following components (INCI designations): hydroxypropylcellulose, PVP, Vinyl alcohol/butyl maleate/isobornyl acrylate copolymer, methyl hydroxypropylcellulose, hydroxypropylguar, polyacrylate crosspolymer-6, ammonium acryloyldimethyltaurate/VP copolymer, carbomer, acrylate copolymer, hydroxyethylacrylate/sodium acryloyldimethyl taurate copolymer, acrylamide/sodiumacryloyldimethyl taurate, acrylate/C10-C30 alkyl acrylate crosspolymer, acrylate/vinyl isodecanoate crosspolymer and their mixtures.

According to a particular embodiment, the composition according to the invention, advantageously, in the oily phase, comprises at least one natural or chemical molecule with specific properties in virology, such as glycyrrhizin, baicalin, quercetin, theaflavin, or their respective derivatives.

It emerges from this that the oily phase of the composition according to the invention can comprise all of the combinations of the components listed above. In other words, the oily phase according to the invention can comprise at least one vegetable oil and/or at least one fat or oil and/or at least one polymer and/or at least one purified vegetable substance with specific virology properties.

According to a particular embodiment, the eco-biological, lipo-alcoholic composition according to the invention comprises at least 12% by total weight of the oily phase composition, advantageously at least 20%.

According to a particular embodiment, when the composition according to the invention comprises at least 80% w/w of C₁-C₈ alcohol, it comprises at least 11% total weight of the oily phase composition.

According to another embodiment, when the composition according to the invention comprises between 75% and strictly less than 80% w/w of C₁-C₈ alcohol, it comprises between 10% and 30% total weight of the composition of the oily phase, advantageously between 20% and 30%.

According to a particular embodiment, the composition according to the invention also comprises:
at least one lipophilic component, advantageously a lipophilic component chosen from among the natural lipophilic constituents of skin tissue, and/or biomimetic lipophilic components; and/or
at least one hydrophilic component, advantageously a hydrophilic component chosen from among the natural hydrophilic constituents of skin tissue, and/or biomimetic hydrophilic components; and/or
at least one component chosen from among the surfactants, gelling agents, dyes of the aqueous phase and/or of the oily phase, and/or
from pure hydrogen peroxide at a concentration less than 1% w/w.

According to the invention, “biomimetic component” means a component that has a structure close to a natural skin component and that makes it possible to activate the same effectors as said skin compound or product; or that mimic the action of a component naturally present in the skin, with the goal of producing the same effects.

According to a particular embodiment, the composition according to the invention has a pH between 5 and 7.5; advantageously between 5.5 and 6.8.

According to a particular embodiment, the eco-biologic, lipo-alcoholic composition according to the invention is a bi-phasic composition in the form of two immiscible phases each present in a continuous form and free of surfactants.

According to another embodiment, the eco-biologic, lipo-alcoholic composition according to the invention is in the form of a gel and comprises a gelling agent representing, advantageously, between 0.1% and 5% total weight of the composition.

Advantageously, the gelling agent is chosen from among the list of the following components (INCI designations): Sodium Hyaluronate, Xanthan gum, Cellulose Gum, Sodium Acrylates Copolymer, Algin, Acrylates/Vinyl Isodecanoate Crosspolymer, Sodium Acrylate/Sodium Acryloyldimethyl Taurate Copolymer, Acrylates/C10-30 Alkyl Acrylate Crosspolymer, Disteardimonium Hectorite, Hydroxyethyl Acrylate/Sodium Acryloyldimethyl Taurate Copolymer, Microcrystalline Cellulose, Sclerotium Gum and their mixtures.

According to another particular embodiment, the eco-biologic lipo-alcoholic composition according to the invention is in the form of an emulsion.

According to a particular embodiment, the eco-biologic lipo-alcoholic composition according to the invention does not contain any of the following components:
perfumes; and
essential oils, advantageously, the essential oils of eucalyptus, tea tree, laurel, rosemary, spearmint, pepper, rose, sage, peppermint, camphor, mukorossi and soapberry; and
terpenes, advantageously, orange terpenes,
silicon-based emulsifiers; and
foaming surfactant type cleaning agents, advantageously ammonium lauryl sulfoccinate and lauramidopropyl betaine; and
pH stabilizers, advantageously lactic acid and pyrogenic silica.

All of these components, well-known to the person skilled in the art for the creation of conventional formulations, cannot be used in the formulation of the composition according to the invention intended to be used without rinsing and repeatedly throughout the day, since, under these use conditions, they cause intolerances that may, under certain circumstances, be very severe, along with secondary effects such as sensitization or multi-sensitization, inflammations causing redness, sores, cracking, etc . . . . Therefore, the problem was to perfect compositions specifically both as effective in terms of virucidal properties, but that might be well-tolerated and not impact the barrier function of the skin.

According to another embodiment, the composition according to the invention is incorporated into a wipe or compress.

The lipo-alcoholic composition according to the invention:
provides micro-biocidal (that is, bactericidal and/or yeasticidal) and/or virucidal effects,
reconstructs and/or maintains and/or improves the lipid barrier function of the skin, and is perfectly well-tolerated by human skin tissues, including during periods of intensive use.

The composition according to the invention is used for disinfecting the skin by elimination of viruses, if applicable, of bacteria and possible of yeasts.

According to the invention, “skin” means any keratinous substrate on the external surface of the body, including, but not limited to, the hands, face, underarms, hair and scalp.

Advantageously, the composition according to the invention is used for disinfecting the hands.

Advantageously, it concerns a non-therapeutic use of the composition according to the invention since it relates to the removal of bacteria, viruses and yeasts from healthy skin, which is not necessarily a prophylactic use since, even in the presence of potentially pathogenic bacteria, viruses and yeasts on the skin, an individual will not necessarily develop a disease.

According to another aspect, the invention relates to a non-therapeutic method for disinfection of the hands consisting of:
providing an eco-biologic, lipo-alcoholic composition without rinsing as described above.
applying the composition to the hands by friction with a contact time of at least 30 seconds, advantageously at least 60 seconds.

According to another aspect, the invention relates to a method for the manufacture of the composition as described above, consisting of:
mixing the oily components in order to produce the oily phase;
adding the C₁-C₈ alcohol, preferably ethanol, advantageously absolute ethanol, to the oily phase prepared in step a).

According to a particular embodiment, step a) also comprises the addition:
of at least one natural lipophile constituent of the skin tissue; and/or
of at least one natural hydrophile constituent of the skin tissue; and/or
of at least one component chosen from among the surfactants, gelling agents, dyes of the aqueous phase and/or of the oily phase; and/or
from pure hydrogen peroxide at a concentration less than 1% w/w.
at least one lipophilic biomimetic component; and/or
at least one hydrophilic biomimetic component.

According to another embodiment, step a) also comprises the addition:
of at least one natural lipophile constituent of the skin tissue; and/or
of at least one natural hydrophile constituent of the skin tissue; and/or
of at least one component chosen from among the surfactants, gelling agents, dyes of the aqueous phase and/or of the oily phase; and/or
from pure hydrogen peroxide at a concentration less than 1%/w/w; and/or
of a gellifying agent, advantageously representing between 0.1% and 5% in total weight of the composition.

Advantageously, the gelling agent is chosen from among the list of the following components (INCI designations): Sodium Hyaluronate, Xanthan gum, Cellulose Gum, Sodium Acrylates Copolymer, Algin, Acrylates/Vinyl Isodecanoate Crosspolymer, Sodium Acrylate/Sodium Acryloyldimethyl Taurate Copolymer, Acrylates/C10-30 Alkyl Acrylate Crosspolymer, Disteardimonium Hectorite, Hydroxyethyl Acrylate/Sodium Acryloyldimethyl Taurate Copolymer, Microcrystalline Cellulose, Sclerotium Gum and their mixtures.

The manufacturing method according to the invention has the advantages of being especially simple, inexpensive, and industrially scalable.

The invention and the advantages resulting from it will be better understood from the following examples given by way of illustration and not limitatively.

### DESCRIPTION OF EXAMPLES OF EMBODIMENTS

### Example 1-Biphasic Cosmetic Compositions According to the Invention

The ingredients are identified using the INCI nomenclature and the percentages are given in weight/weight, in table 1 below:

TABLE 1

| | T | 1A | 1B | 1C | 1D | 1E | 1F |
|---|---|---|---|---|---|---|---|
| Ethanol | 80 | 60 | 65 | 70 | 75 | 80 | 80 |
| Cocoglyceride | — | 30 | 25 | 25 | 23 | 11 | 9 |
| Squalane | — | 5 | 5 | — | 2 | 2 | 2 |
| Glycerol | 2 | — | — | — | — | — | — |
| Water | 18 | 5 | 5 | 5 | — | 7 | 9 |

### Bactericide, Virucide and Yeasticide Effects:

The formulas are tested using the NF EN 13727 standard according to the specific protocol for sanitary friction washing. Briefly, the formulas to be assessed are put into contact with different micro-organisms in suspension after a contact time of 30 and 60 seconds at 20° C., at a test concentration of 50 and 80%.

The minimum test strains assessed are: Pseudomonas aerupinosa, Staphylococcus aureus, Enterococcus hirae, Echerichia coli K12, Candida albicans DSM1386, Poliovirus 1 Sabin strain, Adenovirus type 5, Norovirus murin S99, virus of the Ankara ATCC VR-1508 strain.

If the microorganisms are destroyed, the test is validated and graded “+”, if not all of the microorganisms are destroyed, the result is graded “+/−”, If the microorganisms are not destroyed under the test conditions, the result is graded “−”.

### Skin Barrier Reconstruction Effect:

The in vivo measurement of the transepidermal water loss (TWL) is used in order to assess, in a non-invasive manner, the quality of the barrier function of the stratum corneum.

Series of 20 healthy volunteers were recruited, who used either a commercial hydro-alcoholic gel or solution, or the product of the invention, on a daily basis (studies performed double blind). Before use, then 1 week after, then 1 month after use, the TWL was measured using a Tewameter TM300 (Monaderm), and the results were expressed in % reduction of the TWL (improvement of the barrier effect) in relation to average TWL values obtained with the commercial hydro-alcoholic solution or gel, at each test time.

If the barrier effect improvement results are greater than 50% either at 1 week or at 1 month, the test is graded “+”, if the improvement shows variations depending on the time of measurement, but remains close to 50%, the test is graded “+/−”, if this improvement is less than 50%, the test is graded “−”.

### Tolerance Effect:

During the TWL study above, the assessment of the skin quality (inflammations, irritations, redness, lesions) of the hands of the volunteers who continued using the assessed products several times a day was performed by a dermatologist. If redness or intolerance was observed in more than 30% of the hands assessed, the result was graded “−”, if these were observed on more than 10% of the hands assessed, the result was graded “+/−”, if these were observed on less than 10% of hands assessed, the result was graded “−”.

### Results:

The results are described in table 2.

TABLE 2

| | T | 1A | 1B | 1C | 1D | 1E | 1F |
|---|---|---|---|---|---|---|---|
| Bactericide | + | +/− | + | + | + | + | + |
| Virucide | + | +/− | + | + | + | + | + |
| Yeasticide | + | +/− | + | + | + | + | + |
| Barrier Effect | − | + | + | + | + | + | +/− |
| Tolerance | −−−− | + | + | + | + | + | +/− |

The formulations from example 1, in particular examples 1B to 1E, are therefore capable of exercising all of the expected effects, contrary to the currently marketed hydro-alcoholic solutions. The virucide effect is obtained beginning with 65% of alcohol. The barrier effect is obtained with more than 10% lipids. Tolerance varies with the rate of lipids contributed by the formulation. Cl Example 2-Biphasic Cosmetic Compositions

The ingredients are identified using the INCI nomenclature and the percentages are given in weight/weight in table 3.

TABLE 3

| | T | 2A | 2B | 2C | 2D | 12E | 2F |
|---|---|---|---|---|---|---|---|
| Ethanol | 80 | 65 | 70 | 70 | 75 | 80 | 85 |
| C12-C15 aIkyI- | — | 20 | — | 25 | — | 9 | 5 |
| benzoate | | | | | | | |
| Butyrospermum | — | — | 20 | — | 21 | 18 | 5 |
| parkii oil | | | | | | | |
| Squalane | — | 5 | 5 | — | 2 | 2 | 2 |
| Glycerol | 2 | — | — | — | — | — | — |
| Water | 18 | 10 | 5 | 5 | 2 | 1 | 3 |

The measurements of the bactericide, virucide, yeasticide and tolerance effects are performed according to the protocols described in example 1 of the invention.

### Results:

The results are described in table 4.

TABLE 4

| | T | 2A | 2B | 2C | 2D | 2E | 2F |
|---|---|---|---|---|---|---|---|
| Bactericide | + | + | + | + | + | + | + |
| Virucide | + | + | + | + | + | + | + |
| Yeasticide | + | +/− | + | + | + | + | + |
| Barrier effect | − | + | + | + | + | + | + |
| Tolerance | −−−− | + | + | + | + | + | +l− |

The formulations from example 2, in particular examples 2B to 2E, are therefore capable of exercising all of the expected effects, contrary to the currently marketed hydro-alcoholic solutions.

### Example 3-Biphasic Cosmetic Compositions

The ingredients are identified using the INCI nomenclature and the percentages are given in weight/weight in table 5.

TABLE 5

| | T | 3A | 3B | 3C | 3D | 3E | 3F |
|---|---|---|---|---|---|---|---|
| Isopropanol | 80 | 60 | 65 | 70 | 75 | 80 | 85 |
| Dicaprylyl | — | 30 | 25 | 25 | 21 | 13 | 10 |
| carbonate | | | | | | | |
| Squalane | — | 5 | 5 | — | 2 | 2 | 2 |
| Glycerol | 2 | — | — | — | — | — | — |
| Water | 18 | 5 | 5 | 5 | 2 | 5 | 3 |

The measurements of the bactericide, virucide, yeasticide, and tolerance effects are performed according to the protocols described in example 1 of the invention.

### Results:

The results are described in table 6.

TABLE 6

| | T | 3A | 3B | 3C | 3D | 3E | 3F |
|---|---|---|---|---|---|---|---|
| Bactericide | + | +/− | + | + | + | + | + |
| Virucide | + | +/− | + | + | + | + | + |
| Yeasticide | + | + | + | + | + | + | + |
| Barrier effect | − | + | + | + | + | + | + |
| Tolerance | −−−− | + | + | + | + | + | +l− |

The formulations from example 3, in particular examples 3B to 3E, are therefore capable of exercising all of the expected effects, contrary to the currently marketed hydro-alcoholic solutions.

### Example 4-Biphasic Cosmetic Compositions

The ingredients are identified using the INCI nomenclature and the percentages are given in weight/weight in table 7.

TABLE 7

| | T | 4A | 4B | 4C | 4D | 4E | 4F |
|---|---|---|---|---|---|---|---|
| Ethanol | 80 | 65 | 70 | 70 | 75 | 75 | 80 |
| Dimethicone | — | 30 | 15 | — | 23 | — | 9 |
| Caprylyl | — | — | 12 | 27 | — | 23 | 10 |
| Methicone | | | | | | | |
| Glycerol | 2 | — | — | — | — | — | — |
| Water | 18 | 5 | 3 | 3 | 2 | 2 | 1 |

The measurements of the bactericide, virucide, yeasticide and tolerance effects are performed according to the protocols described in example 1 of the invention.

### Results

The results are described in table 8.

TABLE 8

| | T | 4A | 4B | 4C | 4D | 4E | 4F |
|---|---|---|---|---|---|---|---|
| Bactericide | + | + | + | + | + | + | + |
| Virucide | + | + | + | + | + | + | + |
| Yeasticide | + | + | + | + | + | + | + |
| Barrier effect | − | + | + | + | + | + | + |
| Tolerance | −−−− | + | + | + | + | + | + |

The formulations from example 4, in particular examples 4B to 4F, are therefore capable of exercising all of the expected effects, contrary to the currently marketed hydro-alcoholic solutions.

### Example 5-Cosmetic Compositions of Lipo-Alcoholic Gels

The ingredients are identified using the INCI nomenclature and the percentages are given in weight/weight in table 9.

TABLE 9

| | T | 5A | 5B | 5C | 5D | 5E | 5F |
|---|---|---|---|---|---|---|---|
| Ethanol | 75 | 75 | 75 | 75 | 75 | 75 | 75 |
| Caprylic/caprictriglyceride | — | 22 | 2 | 19 | 19 | 18 | 18 |
| Squalane | — | 2 | 20 | 2 | 2 | 2 | 2 |
| Glycerol | 2 | — | — | — | — | — | — |
| Water | 23 | 2 | 2 | 2 | 2 | 2 | 2 |
| Hydroxypropylcellulose | — | 1 | 1 | — | — | — | 1 |
| PVP | — | — | — | 2 | — | — | — |
| VA/Butyl Maleate/IsobornyI- | — | — | — | — | 2 | — | — |
| Acrylate Copolymer | | | | | | | |
| MethylHydroxypropylcellulose | — | — | — | — | — | 3 | |
| Hydroxypropylguar | — | — | — | — | — | — | 2 |

The measurements of the bactericide, virucide, yeasticide and tolerance effects are performed according to the protocols described in example 1 of the invention.

### Results:

The results are described in table 10.

TABLE 10

| | T | 5A | 5B | 5C | 5D | 5E | 5F |
|---|---|---|---|---|---|---|---|
| Bactericide | + | + | + | + | + | + | + |
| Virucide | + | + | + | + | + | + | + |
| Yeasticide | + | + | + | + | + | + | + |
| Barrier effect | − | + | + | + | + | + | + |
| Tolerance | −−−− | + | + | + | + | + | + |

The formulations from example 5 are therefore capable of exercising all of the expected effects, contrary to the currently marketed hydro-alcoholic solutions.

### Example 6-Cosmetic Compositions of Lipo-Alcoholic Emulsions

The ingredients are identified using the INCI nomenclature and the

percentages are given in weight/weight in table 11.

TABLE 11

| | T | 6A | 6B | 6C | 6D | 6E | 6F |
|---|---|---|---|---|---|---|---|
| Ethanol | 75 | 75 | 80 | 75 | 80 | 75 | 80 |
| Caprylic/capric triglyceride- | — | 14 | 10 | 14 | 10 | 14 | 10 |
| Squalane | — | 2 | 2 | 2 | 2 | 2 | 2 |
| Glycerol | 2 | — | — | — | — | — | — |
| Water | 23 | 5 | 5 | 5 | 5 | 5 | 5 |
| Polyglyceryl 10 Isostearate | — | 4 | 3 | — | — | — | — |
| Polyglyceryl 10 Laurate | — | — | — | 4 | 3 | — | — |
| Sucrose Stearate | — | — | — | — | — | 4 | 3 |

The measurements of the bactericide, virucide, yeasticide and tolerance effects are performed according to the protocols described in example 1 of the invention.

### Results:

The results are described in table 12.

TABLE 12

| | T | 6A | 6B | 6C | 6D | 6E | 6F |
|---|---|---|---|---|---|---|---|
| Bactericide | + | + | + | + | + | + | + |
| Virucide | + | + | + | + | + | + | + |
| Yeasticide | + | + | + | + | + | + | + |
| Barrier effect | − | + | + | + | + | + | + |
| Tolerance | −−−− | + | + | + | + | + | + |

The formulations from example 6 are therefore capable of exercising all of the expected effects, contrary to the currently marketed hydro-alcoholic solutions.

### Example 7-Cosmetic Compositions According to the Invention

The ingredients are identified using the INCI nomenclature and the percentages are given in weight/weight in tables 13 and 14. T corresponds to a formulation of the composition according to the invention and formulations 7A to 7N are formulations from the prior art.

TABLE 13

| | T | 7A | 7B | 7C | 7D | 7E | 7F | 7G |
|---|---|---|---|---|---|---|---|---|
| Ethanol | 75 | 75 | 75 | 75 | 75 | 75 | 75 | 75 |
| Caprylic/capric | 12 | 12 | 12 | 12 | 12 | 12 | 12 | 12 |
| triglyceride | | | | | | | | |
| Water | 13 | 11 | 11 | 11 | 12.5 | 12 | 12 | 12 |
| Phenoxyethanol | — | 2 | — | — | — | — | — | — |
| Benzalkonium Chloride | — | — | 2 | — | — | — | — | — |
| Perfume | — | — | — | 2 | — | — | — | — |
| Terpenoide (Cineole) | — | — | — | — | 0.5 | — | — | — |
| Eucalyptus Oil | — | — | — | — | — | 1 | — | — |
| Peppermint Essential Oil | — | — | — | — | — | — | 1 | — |
| Mukurossi Essential Oil | — | — | — | — | — | — | — | 1 |

TABLE 14

| | T | 7H | 7I | 7J | 7K | 7L | 7M | 7N |
|---|---|---|---|---|---|---|---|---|
| Ethanol | 75 | 75 | 75 | 75 | 75 | 75 | 75 | 75 |
| Caprylic/capric | 12 | 12 | 12 | 12 | 12 | 12 | 12 | 12 |
| triglyceride | | | | | | | | |
| Water | 13 | 3 | 8 | 5 | 10 | 8 | 5 | 5 |
| Fumed silica | — | 10 | — | 5 | — | — | — | — |
| Lactic Acid | — | — | 5 | 3 | — | — | — | — |
| PEG/PPG dimethicone | — | — | — | — | 3 | 5 | — | — |
| Ammonium lauryl- | — | — | — | — | — | — | 8 | — |
| sulfoccinate | | | | | | | | |
| Lauramidopropyl betaine | — | — | — | — | — | — | — | 8 |

The measurements of the bactericide, virucide, yeasticide and tolerance effects are performed according to the protocols described in example 1 of the invention.

### Results:

The results are described in tables 15 and 16.

TABLE 15

| | T | 7A | 7B | 7C | 7D | 7E | 7F | 7G |
|---|---|---|---|---|---|---|---|---|
| Bactericide | + | + | + | + | + | + | + | + |
| Virucide | + | + | + | + | + | + | + | + |
| Yeasticide | + | + | + | + | + | + | + | + |
| Barrier effect | + | + | − | − | + | + | − | − |
| Tolerance | + | − | −− | −− | −− | −− | −− | −− |

TABLE 16

| | T | 7H | 7I | 7J | 7K | 7L | 7M | 7N |
|---|---|---|---|---|---|---|---|---|
| Bactericide | + | + | + | + | + | + | + | + |
| Virucide | + | + | + | + | + | + | + | + |
| Yeasticide | + | + | + | + | + | + | + | + |
| Barrier effect | + | − | − | + | + | + | − | − |
| Tolerance | + | −− | −− | −− | −− | −− | −− | −− |

Therefore, the formulations from example 7 (7A to 7N) are not capable of exercising all of the expected effects of the invention, contrary to control T, which corresponds to a composition according to the invention. Strong to very strong intolerance problems were only revealed after several days' multiple-times-a-day applications, in particular with the use of essential oils, perfumes, pH regulators, bactericides, and in an even more surprising way, with emulsions derived from silicon and with foaming surfactants.

1-15. (canceled).

16. An eco-biologic, lipo-alcoholic topical disinfectant composition, comprising:
at least 65% w/w of a C₁-C₈ alcohol; and
at least 10% by total weight of the composition of an oily phase comprising at least one component selected from the group consisting of plant oils, mineral oils, silicone oils, butters, esters, plant waxes, mineral waxes, silicone waxes, fatty acids, and fatty alcohols with fatty chains of more than 10 carbon atoms in length,
wherein the composition is virucidal, bactericidal, and/or yeasticidal,
wherein the composition is capable of regenerating the skin lipid barrier and is well tolerated by the skin.

17. The eco-biologic, lipo-alcoholic topical composition according to claim 16, wherein the C₁-C₈ alcohol is ethanol, isopropanol, propanol, butanol, pentanol, hexanol, heptanol, octanol, and a mixture thereof, wherein the C₁-C₈ alcohol is in linear or branched form.

18. The eco-biologic, lipo-alcoholic topical composition according to claim 16, wherein the composition further comprises water, and wherein the water is present in an amount less than the amount of alcohol and less than the amount of the oily phase.

19. The eco-biologic, lipo-alcoholic topical composition according to claim 16, wherein the C₁-C₈ alcohol is present in an amount between 65% and 80% w/w.

20. The eco-biologic, lipo-alcoholic topical composition according to claim 16, wherein the C₁-C₈ alcohol is present in an amount between 70% and 80% w/w.

21. The eco-biologic, lipo-alcoholic topical composition according to claim 16, wherein the oily phase is present in an amount of at least 12% of total weight of the composition.

22. The eco-biologic, lipo-alcoholic topical composition according to claim 16, wherein the oily phase is present in an amount of at least 20%.

23. The eco-biologic, lipo-alcoholic topical composition according to claim 16, wherein the C₁-C₈ alcohol is present in an amount between 75% and 80% w/w, and wherein the oily phase is present in an amount between 10% and 30% total weight of the composition.

24. The eco-biologic, lipo-alcoholic topical composition according to claim 23, wherein the of the oily phase is present in an amount between 20% and 30%.

25. The eco-biologic, lipo-alcoholic topical composition according to claim 16, wherein the C₁-C₈ alcohol is present in an amount of at least 80% w/w of C₁-C₈ alcohol, and wherein the oily phase is present in an amount of at least 11% of total weight of the composition.

26. The eco-biologic, lipo-alcoholic topical composition according to claim 16, further comprising:
at least one lipophilic component; and/or
at least one hydrophilic component;
at least one of a surfactant, gelling agent, dye of the aqueous phase, or dye of the oily phase; and/or
pure hydrogen peroxide at a concentration of less than 1% w/w.

27. The eco-biologic, lipo-alcoholic topical composition according to claim 26, wherein the lipophilic component is a natural lipophilic constituents of skin tissue, and/or a biomimetic lipophilic components

28. The eco-biologic, lipo-alcoholic topical composition according to claim 26, wherein the hydrophilic is a natural hydrophilic constituents of skin tissue, and/or a biomimetic hydrophilic components

29. The eco-biologic, lipo-alcoholic topical composition according to claim 16, wherein the composition has a pH between 5 and 7.5.

30. The eco-biologic, lipo-alcoholic topical composition according to claim 16, wherein the composition is a biphasic composition in the form of two immiscible phases, each present in a continuous form and free of surfactants.

31. The eco-biologic, lipo-alcoholic topical composition according to claim 16, wherein the composition is in the form of a gel and comprises a gelling agent in an amount between 0.1% and 5% in weight of the composition.

32. The eco-biologic, lipo-alcoholic topical composition according to claim 31, wherein the gelling agent is Sodium Hyaluronate, Xanthan gum, Cellulose Gum, Sodium Acrylates Copolymer, Algin, Acrylates/Vinyl Isodecanoate Crosspolymer, Sodium Acrylate/Sodium Acryloyldimethyl Taurate Copolymer, Acrylates/C10-30 Alkyl Acrylate Crosspolymer, Disteardimonium Hectorite, Hydroxyethyl Acrylate/Sodium Acryloyldimethyl Taurate Copolymer, Microcrystalline Cellulose, Sclerotium Gum, or a mixture thereof.

33. The eco-biologic, lipo-alcoholic topical composition according to claim 16, wherein the composition is in the form of an emulsion.

34. The eco-biologic, lipo-alcoholic topical composition according to claim 16, wherein the composition does not contain any of the following components:
perfumes;
essential oils of eucalyptus, tea tree, laurel, rosemary, spearmint, pepper, rose, sage, peppermint, camphor, mukorossi, or soapberry;
terpenes, orange terpenes, or silicon-based surfactants;
foaming surfactants, ammonium lauryl sulfosuccinate, or lauramidopropyl betaine; or
pH stabilizers, lactic acid, or pyrogenic silica.

35. The eco-biologic, lipo-alcoholic topical composition according to claim 16, wherein the composition comprises at least one natural or chemical molecule with specific properties in virology, comprising glycyrrhizin, baicalin, quercetin, theaflavin, or their respective derivatives.
